(19) United States
(12) Plant Patent Application Publication
(10) Pub. No.: US 2018/0153082 P1
(43) Pub. Date: May 31, 2018

(54) Sansevieria plant named 'SUPSAN1608'

(50) ;

(71) Applicant: Suphatchatwong Innovation Co., Ltd., Chonburi (TH)

(72) Inventor: Thumrong Suphachadiwong, Bangphra (TH)

(73) Assignee: Suphatchatwong Innovation Co., Ltd., Chonburi (TH)

(21) Appl. No.: 15/732,575

(22) Filed: Nov. 29, 2017

(30) Foreign Application Priority Data
Nov. 30, 2016 (QZ) 2016/3007

(51) Int. Cl. A01H 5/12

(52) U.S. Cl. PLT/382

(57) ABSTRACT

A new and distinct Sansevieria plant named ‘SUPSAN1608’ which is characterized by the combination of a compact growth habit, broad orbicular plant form, thick succulent foliage with a narrow ovate to lanceolate shape, yellow-green foliage with a silver hue and prominent green mottled radial bands, and the stability of all characteristics from generation to generation.

### CROSS REFERENCE TO RELATED APPLICATIONS

This application claims priority to the Community Plant Variety Rights application number 2016/3007, filed Nov. 30, 2016, which is herein incorporated by reference.

Latin name of the genus and species: The Latin name of the genus and species of the novel variety disclosed herein is Sansevieria cylindrica.

Variety denomination: The inventive variety of Sansevieria disclosed herein has been given the variety denomination ‘SUPSAN1608’.

### BACKGROUND OF THE INVENTION

Parentage: The Sansevieria variety ‘SUPSAN1608’ originated as a seedling selection from the controlled pollination of the proprietary seed parent, Sansevieria cylindrica ‘MXJRM0061’ (not patented), with the proprietary pollen parent, Sansevieria cylindrica ‘MXU1806H61’ (not patented). Both parents were developed by, and are owned by, the inventor and were never commercially released. Said crossing was conducted by the inventor in a small, netted greenhouse in Chonburi, Thailand in December of 2011. Seeds from said cross were harvested, then germinated, and the resulting seedlings were grown to a mature size in order to evaluate for a desirable combination of commercial characteristics. In August of 2013, one candidate plant was observed to exhibit a unique growth habit and leaf coloration. After confirmation of the distinctness and stability of the characteristics first observed, the inventor selected the new Sansevieria cultivar, ‘SUPSAN1608’, for commercial introduction.

Asexual Reproduction: Asexual reproduction of ‘SUPSAN1608’ by way of harvesting vegetative ground shoots was first initiated in August of 2013 at Chonburi, Thailand. Access to all plants was restricted, as plants were kept in a greenhouse not open to the public. Through subsequent propagation of vegetative ground shoots, five generations have been reproduced which have shown that the unique features of this cultivar are stable and reproduced true to type.

### SUMMARY OF THE INVENTION

The cultivar ‘SUPSAN1608’ has not been observed under all possible environmental conditions and the phenotype may vary somewhat with variations in environment such as temperature, day length, and light intensity, without, however, any variance in genotype. The following traits have been repeatedly observed and are determined to be the unique characteristics of ‘SUPSAN1608’. These characteristics in combination distinguish ‘SUPSAN1608’ as a new and distinct Sansevieria cultivar:

1. Sansevieria ‘SUPSAN1608’ exhibits a compact, equitant, and upright to outward growth habit; and

2. Sansevieria ‘SUPSAN1608’ exhibits a vertically flattened, broad orbicular plant form; and

3. Sansevieria‘SUPSAN1608’ exhibits thick, succulent foliage with a narrow ovate to lanceolate shape and terete cross-section; and

4. Sansevieria ‘SUPSAN1608’ exhibits yellow-green foliage with a silver hue and prominent green mottled radial bands.

### BRIEF DESCRIPTION OF THE FIGURES

FIG. 1 illustrates, as nearly true as it is reasonably possible to make the same in color photographs of this type, an exemplary plant of ‘SUPSAN1608’ grown in a commercial greenhouse in Chonburi, Thailand. This plant is approximately 12 months old, shown planted in an 8 cm container.

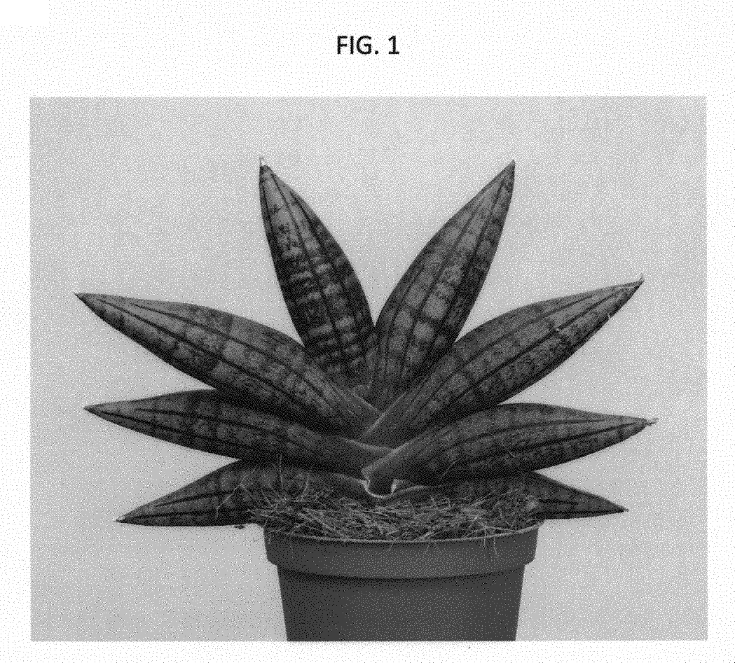

FIG. 2 illustrates, as nearly true as it is reasonably possible to make the same in color photographs of this type, the typical foliage of ‘SUPSAN1608’.

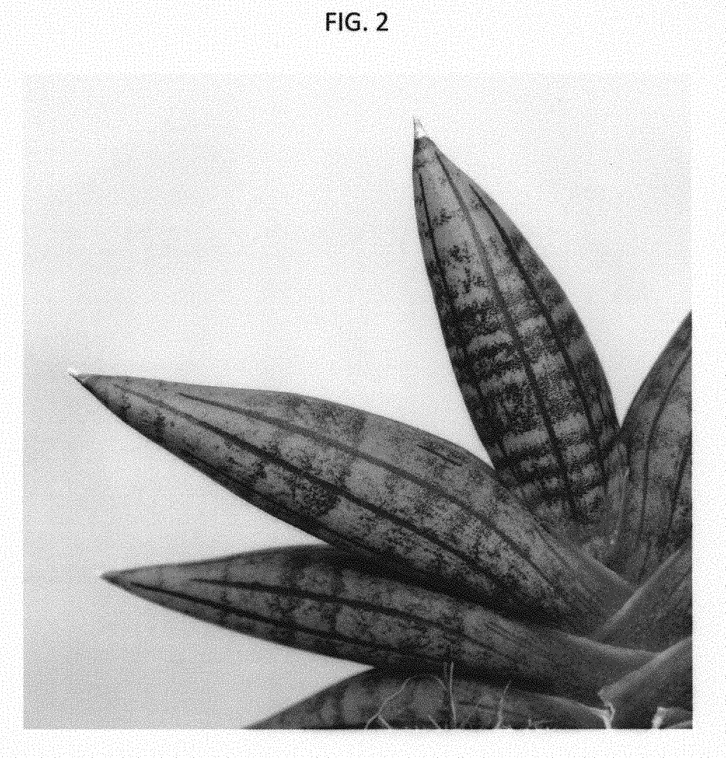

### BOTANICAL DESCRIPTION OF THE PLANT

The following observations and measurements, made in October of 2017, describe averages from a sample set of six specimens of 12 month old ‘SUPSAN1608’ plants grown in 8 cm containers, at a commercial greenhouse in Chonburi, Thailand. The plants were grown using conventional greenhouse production protocols for Sansevieria plants which consisted of overhead irrigation, 50% shade cloth, and no fertilizer. No photoperiodic or chemical treatments were given to the plants.

Those skilled in the art will appreciate that certain characteristics will vary with older or, conversely, with younger plants. ‘SUPSAN1608’ has not been observed under all possible environmental conditions. Where dimensions, sizes, colors and other characteristics are given, it is to be understood that such characteristics are approximations or averages set forth as accurately as practicable. The phenotype of the variety may differ from the descriptions set forth herein with variations in environmental, climatic and cultural conditions. Color notations are based on The Royal Horticultural Society Colour Chart, The Royal Horticultural Society, London, 2015 (sixth edition).

A botanical description of ‘SUPSAN1608’ and comparisons with the parents and most similar commercial variety of Sansevieria are provided below.
Plant description:
   Growth habit.—Monopodial perennial; equitant and upright to outward.
   Plant form.—Vertically flattened, fan-shaped; broad orbicular.
   Average height.—10.0 cm.
   Plant spread.—3.4 cm at the narrowest point, and 15.5 cm at the widest point.
   Plant vigor.—Low.
   Growth rate.—Moderate.
   Propagation type.—Division of vegetative ground shoots; harvesting ground shoots from the mother plant.
   Propagation details.—The time needed to root a shoot division is approximately 2 to 3 months with temperatures ranging from approximately 17 to 40 degrees Celsius.
   Disease resistance.—Plants have not been observed to be susceptible or resistant to pathogens and pests common to Sansevieria.
   Environmental tolerances.—Adapt to, at least, USDA Zones 10 and 12 and temperatures as high as 40 degrees Celsius; moderate tolerance to rain; moderate to high tolerance to wind.
Root system:
   General.—Moderately dense and moderately branched rooting; roots are slightly fibrous.
   Distribution in the soil profile.—Shallow to moderately deep.
   Diameter of roots.—0.15 cm on average.
   Texture.—Smooth; no root hairs.
   Color.—Greyed-yellow, nearest to in between RHS 161B and 161C.
Stem:
   Branching habit.—Monopodial, equitant; decurrent leaf bases form the stem.
   Number of primary (main) stems per plant.—One.
   Number of secondary (lateral) branches per plant.—None.
   Appearance and shape.—Stem is not visible; decurrent leaf bases form the stem.
   Length.—The decurrent leaf bases collectively have an average length of 4.2 cm.
   Diameter.—The decurrent leaf bases collectively have an average diameter of 1.8 cm.
   Internode length.—0.5 cm.
Foliage:
   Arrangement.—Distichous.
   Attachment.—Decurrent.
   Division.—Simple.
   Quantity.—9.
   Attitude.—Outward; at an average angle of 70 degrees to vertical.
   Lamina.—Dimensions — 8.5 cm long and 1.8 cm wide. Thickness — Approximately 1.8 cm. Shape of blade — Narrow ovate to lanceolate; succulent. Cross-section — Terete. Aspect — Nearly straight. Apex — Acute with a slightly papery tip on older leaves, which is colored greyed-white, nearest to RHS 156D. Base — Decurrent. Margin — Leaves are terete so there are no margins, with the exception of the leaf base; leaf base margins are entire. Texture of upper surface — Glabrous, smooth, and succulent. Texture of lower surface — Glabrous, smooth, and succulent. Luster of the upper surface — Matte. Luster of the lower surface — Matte. Color — Juvenile foliage, upper surface — Green, nearest to RHS 138C, with mottled radial bands colored green, nearest to RHS 139A. Juvenile foliage, lower surface — Green, nearest to RHS 138C, with mottled radial bands colored green, nearest to RHS 139A; the base is green, nearest to RHS 143C. Mature foliage, upper surface — Yellow-green, nearest to RHS 146B, with mottled radial bands colored green, nearest to RHS 139A. Mature foliage, lower surface — Yellow-green, nearest to RHS 147D, with mottled radial bands colored green, nearest to RHS 139A; the base is green, nearest to in between RHS 137A and 137B; margins of the decurrent base are a translucent yellow-green, nearest to RHS 147D. Venation — Pattern — Parallel. Color, upper surface — Green, nearest to RHS N189A. Color, lower surface — Green, nearest to RHS N189A.
   Petiole.—No petioles present, leaves are decurrent.

### INFLORESCENCE

No flowering has been observed to date.

### COMPARISONS WITH THE PARENT PLANTS

Plants of the new cultivar ‘SUPSAN1608’ differ from the seed parent, Sansevieria cylindrica ‘MXJRM0061’ (not patented), by the characteristics described in Table 1.

TABLE 1

| Characteristic | ‘SUPSAN1608’ | ‘MXJRM0061’ |
|---|---|---|
| Growth habit. | More compact than | Less compact than |
| | ‘MXJRM0061’. | ‘SUPSAN1608’. |
| Foliage length. | Shorter than | Longer than |
| | ‘MXJRM0061’. | ‘SUPSAN1608’. |
| Foliage; apex. | More acute than | Less acute than |
| | ‘MXJRM0061’. | ‘SUPSAN1608’. |
| General coloration | Darker yellow- | Lighter yellow- |
| of the foliage. | green. | green. |
| Prominence of | More prominent | Less prominent |
| the color pattern | green mottled | green mottled |
| on the foliage. | radial bands. | radial bands. |

Plants of the new cultivar ‘SUPSAN1608’ differ from the pollen parent, Sansevieria cylindrica ‘MXU1806H61’ (not patented), by the characteristics described in Table 2.

TABLE 2

| Characteristic | ‘SUPSAN1608’ | ‘MXU1806H61’ |
|---|---|---|
| Growth habit. | More compact than | Less compact than |
| | ‘MXU1806H61’. | ‘SUPSAN1608’. |
| Rate of growth. | Slower growing than | Faster growing than |
| | ‘MXU1806H61’. | ‘SUPSAN1608’. |
| Leaf length. | Shorter than | Longer than |
| | ‘MXU1806H61’. | ‘SUPSAN1608’. |
| General coloration | Darker green | Lighter green |
| of the foliage | mottled radial . | mottle dradial |
| pattern. | bands | bands. |

### COMPARISON WITH THE MOST SIMILAR SANSEVIERIA CULTIVAR KNOWN TO THE INVENTOR

Plants of the new cultivar ‘SUPSAN1608’ are most similar to the commercial cultivar, Sansevieria cylindrica ‘SAN201202’ (U.S. Pat. No. 24,457). A comparison of ‘SUPSAN1608’ with ‘SAN201202’ is described in Table 3.

TABLE 3

| Characteristic | ‘SUPSAN1608’ | ‘SAN201202’ |
|---|---|---|
| Growth habit. | More compact than | Less compact than |
| | ‘SAN201202’. | ‘SUPSAN1608’. |
| Plant form. | Less flattened. | More flattened. |
| Foliage shape. | Narrow ovate to | Linear; terete. |
| | lanceolate; terete. | |
| Foliage dimensions. | Shorter and broader | Longer and narrower |
| | than ‘SAN201202’. | than ‘SUPSAN1608’. |
| Foliage thickness. | Thicker than | Thinner than |
| | ‘SAN201202’. | ‘SUPSAN1608’. |
| General coloration | Yellow-green with | Dark green with |
| of the foliage. | a silver hue. | a silver hue. |
| Prominence of leaf | More prominent. | Less prominent. |
| venation. | | |
| Prominence of the color | More prominent green | Less prominent green |
| pattern on the foliage. | mottled radial bands. | mottled radial bands. |

That which is claimed is:

1. A new and distinct variety of Sansevieria plant named ‘SUPSAN1608’, substantially as described and illustrated herein.
